(19) United States
(12) Patent Application Publication
(10) Pub. No.: US 2013/0129680 A1
(43) Pub. Date: May 23, 2013

(54) METHOD FOR TREATING HEPATITIS C VIRUS INFECTION USING QUERCETIN-CONTAINING COMPOSITIONS

(71) Applicant: Thomas Christian Lines, Wayland, MA (US)

(72) Inventor: Thomas Christian Lines, Wayland, MA (US)

(21) Appl. No.: 13/303,467

(22) Filed: Nov. 23, 2011

(51) Int. Cl. A61K 31/375; A61K 31/519; A61K 38/21; A61K 31/7056; A61K 38/08; A61K 38/06; A61P 31/14; A61P 37/04; A61P 9/00; A61P 15/00; A61P 31/12; A61P 1/16; A61P 35/00; A61K 31/122; A61K 31/455

(52) U.S. Cl. 424/85.7; 514/456; 514/356; 514/249; 514/43; 514/4.3; 424/94.1; 514/675

(57) ABSTRACT

A method for treating hepatitis C virus infection with a composition containing quercetin, together with one or more of vitamin B₃, vitamin C, a folate compound. Also disclosed is a method for treating conditions associated with an elevated level of heat shock proteins, including liver cancer, using the above-mentioned composition.

### BACKGROUND

Hepatitis C is an infectious disease caused by the hepatitis C virus (HCV) that primarily affects the liver. The infection is often asymptomatic, but chronic infection can lead to scarring of the liver and ultimately to cirrhosis. Cirrhosis often precedes liver failure or liver cancer. HCV is spread by blood-to-blood contact, most typically during blood transfusion. The majority of patients with chronic HCV infection will not clear it without treatment.

Current therapy for chronic HCV infection includes a combination of pegylated interferon alpha and ribavirin, which results in 76% to 82% sustained virological response (SVR) in patients infected with HCV genotypes 2 and 3. The pegylated interferon alpha/ribavarin regimen typically results in a SVR of only 50% when the patient is infected with HCV genotype 1. The majority of US patients are infected with HCV genotype 1. Two new drugs, i.e., VICTRELIS™ (boceprevir) and INCIVEK™ (telaprevir), each can be used in conjunction with pegylated interferon alpha and/or ribavirin to treat chronic HCV infection. The above-mentioned drug regimens typically lead to many side-effects, including but not limited to fever, fatigue/myalgias, headache, nausea, arthralgias, depression, skin rash, neutropenia, anemia, thrombocytopenia, and birth defects.

It is known that certain natural antioxidants which also have an anti-viral effect, such as quercetin, inhibit both acute and chronic phases of viral illnesses and free-radical induced diseases. Further, some natural antioxidants exhibit synergy in their reactions with biologically relevant oxygen species, e.g., hydroxyl radicals, superoxides, oxysulfurs, sulfur dioxide, and nitrogen dioxide.

Quercetin, in addition to ameliorating free-radical induced diseases, also inhibits the synthesis of heat shock proteins. Heat shock proteins (HSP) are intracellular proteins known as molecular chaperones. HSPs are involved in the proper folding of proteins, as well as the cellular response to injury or stress. In some instances, HSPs are required for viral replication or infection.

### SUMMARY

The present invention features a method for treating HCV infection by administering to a subject in need thereof an effective amount of a quercetin-containing composition, which also includes one or more of vitamin B₃, vitamin C, and a folate compound. In another aspect, the invention features a method for treating HCV infection using a composition containing quercetin, vitamin B3, vitamin C, and folic acid in the form of L-methyl folate (also known as 5-methyltetrahydrofolate or METAFOLIN™). Additionally, another aspect of the invention features a method for treating HCV infection using an anti-viral drug together with the above-mentioned compositions.

In still another aspect, the invention features a method for treating conditions that are caused, in part, by overexpression of heat shock proteins. These conditions include but are not limited to autoimmune diseases, vascular disorders, pregnancy-related disorders, viral infections, and certain cancers. The method relies on administering to a subject in need thereof an effective amount of the above-described compositions.

The composition, either in dry form (e.g., powder or tablet) or in liquid form (e.g., beverage or syrup), can be a dietary supplement or a pharmaceutical formulation. The dietary supplement or the pharmaceutical formulation can be in the form of a tablet, a capsule, a soft chew, a gel, or a sterile injectable solution. The composition can also be a food product. Examples include tea (e.g., a tea drink and the contents of a tea bag), soft drinks, juice (e.g., a fruit extract and a juice drink), milk, coffee, jelly, ice cream, yogurt, cookies, cereals, chocolates, and snack bars.

The details of one or more embodiments of the invention are set forth in the description below. Other features, objects, and advantages of the invention will be apparent from the description and from the claims.

### DETAILED DESCRIPTION

This invention is based, in part, on the unexpected findings that quercetin, together with one or more of vitamin B₃ vitamin C, and a folate compound, exhibits synergistic inhibition of HCV intracellular protein production and infectious virus production in an HCV-infected subject.

Accordingly, the present invention features a method for treating a subject infected by HCV by administering an effective amount of a composition containing quercetin and vitamin B₃. In another embodiment, quercetin and vitamin C may be used to treat HCV infection. Further, HCV can be treated using a composition that includes quercetin, vitamin B₃, and vitamin C. In an additional embodiment, the above mentioned compositions can also contain a folate compound, preferably L-methyl folate. Additionally, luteolin, epigallocatechin gallate (EGCG), or both can be added to the compositions described above. HCV, according to the invention, can also be treated by co-administering the above-described compositions with an anti-viral drug. Co-administration to a subject of the compositions with an anti-viral drug, according to the invention, reduces the side effects associated with the anti-viral drugs (e.g., fever, fatigue/myalgias, headache, nausea, arthralgias, depression, skin rash, neutropenia, anemia, thrombocytopenia, and birth defects) and advantageously allows for a lower dose of these drugs to be used to treat HCV infection. The term “co-administration” refers to simultaneous administration or sequential administration of two different treatment modalities. The phrase “sequential administration” refers to administering a second composition soon after a first composition. For example, the second composition can be administered 30 minutes, 1 h, 2 h, or 4 h after administration of the first composition.

Without being bound by theory, the above described compositions may function to ameliorate HCV infection by the following mechanism.

HCV, like most viruses, depends upon host cell proteins in order to replicate and produce infectious viral particles. Among the host cell proteins required for HCV propagation are heat shock proteins (HSPs). HSPs are intracellular proteins known as molecular chaperones. HSPs are involved in the proper folding of proteins, as well as the cellular response to injury or stress. Quercetin, together with one or more of vitamin B3, vitamin C, and a folate compound, synergistically inhibits the synthesis of heat shock proteins, which are required for HCV propagation.

Diseases that result from HCV infection, e.g., cirrhosis and liver cancer, can be treated with the compositions set forth above, either as a stand-alone treatment or in conjunction with currently accepted therapies. In the case of liver cancer, the above-mentioned compositions can be administered alone or together with chemotherapy drugs, including but not limited to doxorubicin, 5-fluorouracil, cisplatin, paclitaxel, gemcitabine, mitoxantrone, epirubicin, capecitabine, and tamoxifen. The compositions described above can also be used for treating diseases or disorders associated with elevated levels of heat shock proteins or antibodies against heat shock proteins. Among these diseases or disorders are autoimmune diseases (e.g., systemic lupus erythematosis, rheumatoid arthritis, systemic sclerosis, and multiple sclerosis), vascular disorders (e.g., peripheral vascular disease, renal vascular disease, and cerebral small vessel disease), pregnancy-related conditions (e.g., transient hypertension of pregnancy, and preeclampsia), coronary heart disease, and cancer (e.g., breast, endometrial, ovarian, cervical, oral, gastric, liver, pancreatic, colorectal, lung, urinary system, prostate, leukemia, lymphoma, pituitary, adrenal, and skin cancers and nervous system tumors).

In addition, the compositions can also be used to treat subjects infected with a virus whose replication depends upon an elevated level of host heat shock protein expression (e.g., adenovirus, polyoma virus, human papilloma virus, and human immunodeficiency virus). Treatment of subjects in need thereof with the compositions described above can lessen negative side effects caused by replication of the just-mentioned viruses.

The efficacy of quercetin is enhanced by vitamin B₃, vitamin C, or both. For example, a combination of quercetin, vitamin B₃, and vitamin C maintains quercetin levels in plasma up to five times those of quercetin alone or a combination of quercetin and vitamin B₃. Further, a combination of quercetin, vitamin B₃, and vitamin C results in a quercetin half life in plasma twice as long as that of quercetin alone and about one and a half times that of a combination of quercetin and vitamin B₃. See U.S. Pat. Nos. 7,745,486 and 7,745,487. A folate compound, preferably L-methyl folate (also known as 5-methyltetrahydrofolate or METAFOLIN™), improves the efficacy of quercetin, as well as the efficacy of quercetin together with vitamin B₃, vitamin C, or both.

Typically, a subject can be administered, once or periodically per day, with the composition in an amount that provides 20 mg to 3 g (preferably, 250 mg to 1 g) of quercetin. When vitamin B₃, vitamin C, or folic acid is included in a composition of this invention, it is preferred that each dose or serving contain 20 μg-3 g vitamin B₃, 200 μg-3 g vitamin C, or 40-3000 μg of a folate compound.

The term “quercetin” refers to both quercetin aglycon and quercetin derivatives including but not limited to quercetin-3-O-glucoside, quercetin-5-O-glucoside, quercetin-7-O-glucoside, quercetin-9-O-glucoside, quercetin-3-O-rutinoside, quercetin-3-O-[α-rhamnosyl-(1→2)-α-rhamnosyl-(1→6)]-β-glucoside, quercetin-3-O-galactoside, quercetin-7-O-galactoside, quercetin-3-O-rhamnoside, and quercetin-7-O-galactoside. After digestion, quercetin derivatives are converted to quercetin aglycon and other active derivatives, which are absorbed in the body. These quercetin aglycones and other active derivatives can be subsequently sulfated, methylated, glucuronylated, and/or glucosidated, among other modifications. The quantity of quercetin mentioned above refers to that of quercetin aglycon or the quercetin moiety of a quercetin derivative.

Quercetin can be added to the composition either in a pure form or as an ingredient in a mixture (e.g., a plant extract). Examples of commercially available quercetin include QU995 (containing 99.5% quercetin) and QU985 (containing 98.5% quercetin) from Quercegen Pharmaceuticals (Sudbury, Mass.) and Merck KGaA (Germany). “Vitamin B₃” mentioned herein includes vitamin B₃ in its various forms, including niacinamide, nicotinic acid, nicotinamide, inositol hexaniacinate. “Vitamin C” mentioned herein includes vitamin C (i.e., L-ascorbic acid, D-ascorbic acid, or both) and its salts (e.g., sodium ascorbate). “Folate compound” mentioned herein includes vitamin B₉, folate, pteroylglutamic acid, and L-methyl folate (also known as 5-methyltetrahydrofolate or METAFOLIN™). The amount of folate compound in a composition of this invention depends on the amounts of the other ingredients, i.e., quercetin, vitamin B₃, and vitamin C. More specifically, it depends on the intended amounts of all 4 ingredients per dose or serving. It is preferred that each dose or serving contain 100-1200 μg of a folate compound.

The composition of this invention can be in various forms. For example, it can be a soft chew composition that includes quercetin, niacinamide, ascorbic acid, sodium ascorbate, folic acid, sugar, corn syrup, sucralose, soy lecithin, corn starch, glycerin, palm oil, xylitol, carrageenan, FD&C Yellow #6, FD&C Yellow #5, and natural and/or artificial flavors. An exemplary serving of this soft chew composition (5.15 g) includes 250 mg of quercetin, 12.9 mg of vitamin B₃ (i.e., niacinamide), and 382.8 mg of vitamin C (i.e., L-ascorbic acid and sodium ascorbate). A subject can take one to eight servings (e.g., 4 servings) of this soft chew composition daily. The amounts taken can vary depending on, for example, the disorder or condition to be treated and the physical states of the subject. Another exemplary composition of this soft chew includes 5.25 wt % of quercetin, 0.25 wt % of vitamin B₃, and 7.81 wt % of vitamin C (i.e., L-ascorbic acid and sodium ascorbate) plus 200 μg of folic acid per chew.

The composition can further contain one or more active ingredient, such as an isoflavone (e.g., genistein or genistin), curcumin, resveratrol, luteolin, epigallocatechin gallate (EGCG), coenzyme Q10, eicosapentaenoic acid (EPA), and docosahexaenoic acid (DHA). A preferred composition contains luteolin, EGCG, or both, in addition to quercetin, vitamin B₃, vitamin C, and a folate compound. These active ingredients can be added to the composition either in a pure form or as a component in a mixture (e.g., an extract from a plant or an animal). A suitable daily dosage of each of these ingredients can vary depending on, for example, the disorder or condition to be treated and the physical states of the subjects. Exemplary daily dosages of some of these ingredients are: 20-2,500 mg (preferably 250-1,000 mg) of curcumin, 10-1,000 mg (preferably 100-500 mg) of resveratrol, 50-1,000 mg (preferably 100-700mg) of EGCG, 25-300 mg (preferably 50-100 mg) of genistin/genistein, 10-1,000 mg (preferably 100-200 mg) of luteolin, 50-1,000 mg (preferably 70-500 mg) of EPA, and 50-1,000 mg (preferably 80-700 mg) of DHA.

When the above-described composition is in powder form, it can be used conveniently to prepare beverage, paste, jelly, capsules, or tablets. Lactose and corn starch are commonly used as diluents for capsules and as carriers for tablets. Lubricating agents, such as magnesium stearate, are typically included in tablets.

The composition of this invention can be a dietary supplement or a pharmaceutical formulation. As a dietary supplement, additional nutrients, such as minerals or amino acids may be included. A pharmaceutical formulation can be a sterile injectable or infusible solution that contains the composition together with pharmaceutically acceptable excipients. The composition can also be a food product. As used herein, the term “food” broadly refers to any kinds of liquid and solid/semi-solid materials that are used for nourishing humans and animals, for sustaining normal or accelerated growth, or for maintaining stamina or alertness. Examples of human food products include, but are not limited to, tea-based beverages, juice, coffee, milk, jelly, cookies, cereals, chocolates, snack bars, herbal extracts, dairy products (e.g., ice cream, and yogurt), soy bean product (e.g., tofu), and rice products.

The terms “improving,” “enhancing,” “treating,” and “lowering” refer to the administration of an effective amount of a composition of the invention to a subject, who needs to improve one or more of the above-mentioned conditions or has one or more of the just-mentioned disorders, or a symptom or a predisposition of one of more of the disorders or conditions, with the purpose to improve one or more of these conditions, or to prevent, cure, alleviate, relieve, remedy, or ameliorate one or more of these disorders, or the symptoms or the predispositions of one or more of them. The term “administration” covers oral or parenteral delivery to a subject a composition of the invention in any suitable form, e.g., food product, beverage, tablet, capsule, suspension, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. An “effective amount” refers to a dose of the composition that is sufficient to provide a therapeutic benefit (e.g., reducing the levels of HCV in the liver or serum). Both in vivo and in vitro studies can be conducted to determine optimal administration routes and doses.

The compositions described above can be preliminarily screened for their efficacy in treating the above-described conditions by in vitro assays and then confirmed by animal experiments and clinic trials. Other suitable analytical and biological assays are apparent to those of ordinary skill in the art. For example, the effectiveness of the compositions described above can be measured by conducting in vitro viral replication studies.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for treating hepatitis C virus infection, the method comprising administering to a subject in need thereof an effective amount of a composition that includes quercetin and vitamin B3, vitamin C, or a folate compound.

2. The method of claim 1, wherein the composition includes vitamin B3.

3. The method of claim 1, wherein the composition includes vitamin C.

4. The method of claim 1, wherein the composition includes a folate compound.

5. The method of claim 4, wherein the folate compound is L-methyl folate.

6. The method of claim 2, wherein the composition includes vitamin C.

7. The method of claim 6, wherein the composition includes a folate compound.

8. The method of claim 7, wherein the folate compound is L-methyl folate.

9. The method of claim 2, wherein the composition includes a folate compound.

10. The method of claim 3, wherein the composition includes a folate compound.

11. The method of claim 1, wherein the composition further includes one or more of genistein, genistin, curcumin, resveratrol, luteolin, epigallocatechin gallate (EGCG), coenzyme Q10, eicosapentaenoic acid (EPA), and docosahexaenoic acid (DHA).

12. The method of claim 11, wherein the composition includes luteolin or epigallocatechin gallate (EGCG).

13. The method of claim 1, wherein the composition is co-administered with an anti-viral drug.

14. The method of claim 13, wherein the anti-viral drug is pegylated interferon alpha, ribavirin, boceprevir, or telaprevir.

15. A method for treating a disorder associated with an elevated level of a heat shock protein, the method comprising administering to a subject in need thereof an effective amount of a composition that includes quercetin and one or more of vitamin B3, vitamin C, or a folate compound.

16. The method of claim 15, wherein the composition includes vitamin B3.

17. The method of claim 16, wherein the composition includes vitamin C.

18. The method of claim 17, wherein the composition includes a folate compound.

19. The method of claim 18, wherein the folate compound is L-methyl folate.

20. The method of claim 15, wherein the composition further includes one or more of genistein, genistin, curcumin, resveratrol, luteolin, epigallocatechin gallate (EGCG), coenzyme Q10, eicosapentaenoic acid (EPA), and docosahexaenoic acid (DHA).

21. The method of claim 20, wherein the composition includes luteolin or epigallocatechin gallate (EGCG).

22. The method of claim 15, wherein the disorder is an autoimmune disease, a vascular disorder, a pregnancy-related disorder, a viral infection, or liver or other cancer.
